SEQUENCE LISTING (30 sequences)

SEQ ID NO: 1 - DNA, 29903 bp, SARS-CoV-2
  source 1..29903: mol_type = genomic DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 19 bp, synthetic construct
  misc_feature 1..19: note = F3 primer
  source 1..19: mol_type = other DNA
tctggtaaaggccaacaac

SEQ ID NO: 3 - DNA, 14 bp, synthetic construct
  misc_feature 1..14: note = B3 primer
  source 1..14: mol_type = other DNA
gctgaagcgctggg

SEQ ID NO: 4 - DNA, 36 bp, synthetic construct
  misc_feature 1..36: note = FIP primer
  source 1..36: mol_type = other DNA
agtacgtttttgccgaggcaaggccaaactgtcact

SEQ ID NO: 5 - DNA, 39 bp, synthetic construct
  misc_feature 1..39: note = BIP primer
  source 1..39: mol_type = other DNA
gtaacacaagctttcggcagattagttcctggtccccaa

SEQ ID NO: 6 - DNA, 17 bp, synthetic construct
  misc_feature 1..17: note = LF primer
  source 1..17: mol_type = other DNA
aagcctcagcagcagat

SEQ ID NO: 7 - DNA, 18 bp, synthetic construct
  misc_feature 1..18: note = LB primer
  source 1..18: mol_type = other DNA
tccagaacaaacccaagg

SEQ ID NO: 8 - DNA, 24 bp, synthetic construct
  misc_feature 1..24: note = F3 primer
  source 1..24: mol_type = other DNA
ttctatagattagctaatgagtgt

SEQ ID NO: 9 - DNA, 22 bp, synthetic construct
  misc_feature 1..22: note = B3 primer
  source 1..22: mol_type = other DNA
acttatcggcaattttgttacc

SEQ ID NO: 10 - DNA, 46 bp, synthetic construct
  misc_feature 1..46: note = FIP primer
  source 1..46: mol_type = other DNA
gaggttccacctggtttaacatatctcaagtattgagtgaaatggt

SEQ ID NO: 11 - DNA, 46 bp, synthetic construct
  misc_feature 1..46: note = BIP primer
  source 1..46: mol_type = other DNA
caggagatgccacaactgcttatagataaaagtgcattaacattgg

SEQ ID NO: 12 - DNA, 18 bp, synthetic construct
  misc_feature 1..18: note = LF primer
  source 1..18: mol_type = other DNA
gaaccgccacacatgacc

SEQ ID NO: 13 - DNA, 20 bp, synthetic construct
  misc_feature 1..20: note = LB primer
  source 1..20: mol_type = other DNA
catttgtcaagctgtcacgg

SEQ ID NO: 14 - DNA, 18 bp, synthetic construct
  misc_feature 1..18: note = F3 primer
  source 1..18: mol_type = other DNA
tcgcaatggcttgtcttg

SEQ ID NO: 15 - DNA, 20 bp, synthetic construct
  misc_feature 1..20: note = B3 primer
  source 1..20: mol_type = other DNA
gcaatacgaagatgtccacg

SEQ ID NO: 16 - DNA, 33 bp, synthetic construct
  misc_feature 1..33: note = FIP primer
  source 1..33: mol_type = other DNA
cgtacgcgcaaacagtggcttgatgtggctcag

SEQ ID NO: 17 - DNA, 37 bp, synthetic construct
  misc_feature 1..37: note = BIP primer
  source 1..37: mol_type = other DNA
cgttccatgtggtcattcatagaagcggtctggtcag

SEQ ID NO: 18 - DNA, 20 bp, synthetic construct
  misc_feature 1..20: note = LF primer
  source 1..20: mol_type = other DNA
gaaagaagcaatgaagtagc

SEQ ID NO: 19 - DNA, 22 bp, synthetic construct
  misc_feature 1..22: note = LB primer
  source 1..22: mol_type = other DNA
gaaactaacattcttctcaacg

SEQ ID NO: 20 - DNA, 36 bp, synthetic construct
  misc_feature 1..36: note = Probe
  misc_feature 36
  misc_feature 36: note = bodipy FL
  source 1..36: mol_type = other DNA
atccagaaactaacattcttctcaacgtgccactcc

SEQ ID NO: 21 - DNA, 23 bp, synthetic construct
  misc_feature 1..23: note = F3 primer
  source 1..23: mol_type = other DNA
tacttggacaatcaaaaagagtt

SEQ ID NO: 22 - DNA, 24 bp, synthetic construct
  misc_feature 1..24: note = B3 primer
  source 1..24: mol_type = other DNA
gtctgtagtaatgatttgtggttc

SEQ ID NO: 23 - DNA, 22 bp, synthetic construct
  misc_feature 1..22: note = B4 primer
  source 1..22: mol_type = other DNA
tgtctgtagtaatgatttgtgg

SEQ ID NO: 24 - DNA, 42 bp, synthetic construct
  misc_feature 1..42: note = FIP primer
  source 1..42: mol_type = other DNA
cttttcttgtgcagggacactatcatcttatgtccttccctc

SEQ ID NO: 25 - DNA, 38 bp, synthetic construct
  misc_feature 1..38: note = BIP primer
  source 1..38: mol_type = other DNA
tcacaactgctcctgcctgaaacaaagacaccttcacg

SEQ ID NO: 26 - DNA, 21 bp, synthetic construct
  misc_feature 1..21: note = LF primer
  source 1..21: mol_type = other DNA
acatgcaagaagactacacca

SEQ ID NO: 27 - DNA, 21 bp, synthetic construct
  misc_feature 1..21: note = LB primer
  source 1..21: mol_type = other DNA
gatggaaaagcacactttcct

SEQ ID NO: 28 - DNA, 29 bp, synthetic construct
  misc_feature 1..29: note = Probe
  misc_feature 29: note = bodipy FL
  source 1..29: mol_type = other DNA
gcaagaagactacaccatgaggtgctgac

SEQ ID NO: 29 - DNA, 377 bp, synthetic construct
  misc_feature 1..377: note = Artificial Sequnce for detecting SARS-CoV-2 M region
  source 1..377: mol_type = other DNA
gtaatacgactcactatagggatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgc

SEQ ID NO: 30 - DNA, 1593 bp, synthetic construct
  misc_feature 1..1593: note = Artificial Sequnce for detecting SARS-CoV-2 S2 region
  source 1..1593: mol_type = other DNA
gtaatacgactcactatagggaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

(19) United States
(12) Patent Application Publication
(10) Pub. No.: US 2025/0333809 A1
(43) Pub. Date: Oct. 30, 2025

(54) METHOD FOR DETECTING CORONAVIRUS (SARS-CoV-2)

(71) Applicant: EIKEN KAGAKU KABUSHIKI KAISHA, Taito-ku, Tokyo (JP)

(72) Inventors: Kota YOKONO, Shimotsuga-gun, Tochigi (JP); Machiko YAMAMOTO, Shimotsuga-gun, Tochigi (JP); Syota YUKI, Shimotsuga-gun, Tochigi (JP); Ai FUKAYA, Shimotsuga-gun, Tochigi (JP); Yutaka KUBOTA, Shimotsuga-gun, Tochigi (JP)

(21) Appl. No.: 19/264,683

(22) Filed: Jul. 9, 2025

Related U.S. Application Data
(62) Division of application No. 17/905,497, filed on Sep. 1, 2022, now Pat. No. 12,410,488.

(30) Foreign Application Priority Data
Mar. 5, 2020 (JP) 2020-038195
Nov. 18, 2020 (JP) 2020-191630

(51) Int. Cl. C12Q 1/70; C12Q 1/6844; C12Q 1/6888

(52) U.S. Cl. CPC C12Q 1/701; C12Q 1/6844; C12Q 1/6888; C12Q 2600/16

(57) ABSTRACT

Disclosed are oligonucleotide primers that hybridize specifically with any base sequence designed from the base sequences of the N gene, RNA-dependent RNA polymerase gene, M gene, and S gene of SARS-COV-2, a nucleic acid amplification method using said primers, a test method for SARS-COV-2 infection by detection of nucleic acid amplification, and a COVID-19 test kit.

### RELATED PATENT APPLICATIONS

This application is a divisional application of U.S. application Ser. No. 17/905,497, filed on Sep. 1, 2022, which is the National Stage of International Application No. PCT/JP2021/008261, filed on Mar. 3, 2021, and claims the benefit of Japanese Application No. 2020-038195, filed on Mar. 5, 2020 and Japanese Application No. 2020-191630, filed on Nov. 18, 2020. The disclosure of the prior application is incorporated herein by reference.

### SEQUENCE LISTING

This application contains a Sequence Listing which has been submitted electronically in WIPO Standard ST.26 (XML format) and is hereby incorporated by reference in its entirety. Said Sequence Listing copy is titled “FP20-1279-01US-XX_SEQL” and is 71,041 bytes in size.

### TECHNICAL FIELD

The present invention relates to a method for detecting a new coronavirus (SARS-COV-2) discovered in 2019, and more specifically to a method for assisting diagnosis of acute respiratory disease (COVID-19) due to the new 2019 coronavirus utilizing a highly sensitive method for detecting genes.

### BACKGROUND ART

Acute respiratory disease (COVID-19) due to the new 2019 coronavirus was reported in December 2019 as outbreaks of patients with pneumonia of unknown causes in Wuhan City, Hubei Province, China. The cause was confirmed to be a new coronavirus on Jan. 7, 2020, and the following week, infected persons were confirmed in a plurality of countries in addition to China. As of the end of January, the number of infected persons exceeded 10,000, but the spread of the infection did not stop, and the World Health Organization (WHO) declared a Public Health Emergency of International Concern (PHEIC) on January 31 st.

The main clinical symptoms of COVID-19 are fever, cough, and general malaise. When it becomes severe, it causes pneumonia or dysfunction of the lungs, the heart, and the kidneys, which may lead to death. Although the mortality is about 3% which is lower than that of the SARS epidemic from 2002 to 2003, the number of infected persons is much higher than that of SARS. Therefore, the death toll is higher than that of the SARS epidemic.

The new virus (SARS-COV-2) that causes COVID-19 is a betacoronavirus similar to the SARS coronavirus or MERS coronavirus. The genes of the new virus consist of a positive strand of single-stranded RNA and have a length of about 30,000 bases (GENBANK Accession No. MN908947).

The entire sequence of SARS-COV-2 was determined and published about 1 month after the first report of the outbreak of pneumonia in patients. Based on the sequence, methods for detecting SARS-COV-2 through RT-PCR have been developed (Non Patent Literature 1 and others).

However, since these detection methods were developed in a short period of time, some of them have problems in detection accuracy. For example, it has been announced that some of test kits developed by The Centers for Disease Control and Prevention (CDC) have defects (refer to Non Patent Literature 2). In addition, the number of tests required is also enormous due to the rapid spread of infection. For these reasons, a faster and more accurate method for detecting SARS-COV-2 is required.

### CITATION LIST

### Non Patent Literature

[Non Patent Literature 1] National Institute of Infectious Disease: Pathogen Detection Manual 2019-nCOV Ver.2.7 (edited on Feb. 25, 2020), Internet <URL: https://www.niid.go.jp/niid/images/lab-manual/2019-nCoV20200225.pdf>
[Non Patent Literature 2] CDC Tests for COVID-19, retrieved on Feb. 18, 2020, Internet <URL: https://www.cdc.gov/coronavirus/2019-ncov/about/testing.html>

### SUMMARY OF INVENTION

### Technical Problem

An object of the present invention is to detect SARS-COV-2, which is a pathogenic virus of COVID-19, with high sensitivity for diagnosis of COVID-19.

### Solution to Problem

The present inventors have conducted extensive studies to solve the above-described problem, and as a result, they have found that SARS-COV-2 can be detected with high sensitivity by producing an oligonucleotide primer that is selectively hybridized with a sequence specific to SARS-COV-2 and amplifying the sequence specific to SARS-COV-2 through a LAMP method, thus leading to realization of the present invention.

That is, the present invention has the following configuration.

[1] A primer set for detecting SARS-COV-2 virus through amplification by a LAMP method, wherein the primer set consists of at least one designed from regions (a) and (b):
(a) a region from a 28976th base to a 29211th base of SEQ ID NO: 1; and
(b) a region from a 15394th base to a 15595th base of SEQ ID NO: 1.

[2] A primer set for detecting SARS-COV-2 virus through amplification by a LAMP method, wherein the primer set consists of at least one selected from the group consisting of (a1) and (b1):
(a1) a primer set consisting of an F3 primer consisting of SEQ ID NO: 2, a B3 primer consisting of SEQ ID NO: 3, an FIP primer consisting of SEQ ID NO: 4, and a BIP primer consisting of SEQ ID NO: 5; and
(b1) a primer set consisting of an F3 primer consisting of SEQ ID NO: 8, a B3 primer consisting of SEQ ID NO: 9, an FIP primer consisting of SEQ ID NO: 10, and a BIP primer consisting of SEQ ID NO: 11.

[3] A primer set for detecting SARS-COV-2 virus through amplification by a LAMP method, wherein the primer set consists of at least one selected from the group consisting of (a2) and (b2):
(a2) an LF primer consisting of SEQ ID NO: 6 and an LB primer consisting of SEQ ID NO: 7 in addition to the primer set of (a1) of [1]; and
(b2) an LF primer consisting of SEQ ID NO: 12 and an LB primer consisting of SEQ ID NO: 13 in addition to the primer set of (b1) of [1].

[4] A method for detecting SARS-COV-2 virus, comprising performing an amplification reaction through a LAMP method using the primer set of any one of [1] to [3].

[5] A method for testing for COVID-19, comprising detecting amplification of a target nucleic acid region of SARS-COV-2 virus using the primer set of any one of [1] to [3] to test for presence or absence of infection with SARS-COV-2 virus.

[6] A kit comprising the primer set of any one of [1] to [3] in a method for diagnosing COVID-19.

[7] A primer set for detecting SARS-COV-2 virus through amplification by a LAMP method, wherein the primer set consists of at least one designed from regions (c) and (d):
(c) a region from a 26767th base to a 26977th base of SEQ ID NO: 1; and
(d) a region from a 24660th base to a 24916th base of SEQ ID NO: 1.

[8] A primer set for detecting SARS-COV-2 virus through amplification by a LAMP method, wherein the primer set consists of at least one selected from the group consisting of (c1) and (d1):
(c1) a primer set consisting of an F3 primer consisting of SEQ ID NO: 14, a B3 primer consisting of SEQ ID NO: 15, an FIP primer consisting of SEQ ID NO: 16, and a BIP primer consisting of SEQ ID NO: 17; and
(d1) a primer set consisting of an F3 primer consisting of SEQ ID NO: 21, a B3 primer consisting of SEQ ID NO: 22, a B4 primer consisting of SEQ ID NO: 23, an FIP primer consisting of SEQ ID NO: 24, and a BIP primer consisting of SEQ ID NO: 25.

[9] A primer set for detecting SARS-COV-2 virus through amplification by a LAMP method, wherein the primer set consists of at least one selected from the group consisting of (c2) and (d2):
(c2) a primer set consisting of an F3 primer consisting of SEQ ID NO: 14, a B3 primer consisting of SEQ ID NO: 15, an FIP primer consisting of SEQ ID NO: 16, a BIP primer consisting of SEQ ID NO: 17, an LF primer consisting of SEQ ID NO: 18, and an LB primer consisting of SEQ ID NO: 19; and
(d2) a primer set consisting of an F3 primer consisting of SEQ ID NO: 21, a B3 primer consisting of SEQ ID NO: 22, a B4 primer consisting of SEQ ID NO: 23, an FIP primer consisting of SEQ ID NO: 24, a BIP primer consisting of SEQ ID NO: 25, an LF primer consisting of SEQ ID NO: 26, and an LB primer consisting of SEQ ID NO: 27.

[10] A kit for detecting SARS-COV-2 virus through amplification by a LAMP method, comprising the primer set of any one of [7] to [9]; and a fluorescence labeling probe.

[11] The kit of [10], wherein the primer set is a primer set of (c1) or (c2), and the fluorescence labeling probe is a probe consisting of SEQ ID NO: 20.

[12] The kit of [10], wherein the primer set is a primer set of (d1) or (d2), and the fluorescence labeling probe is a probe consisting of SEQ ID NO: 28.

[13] The kit of any one of to [12], which is used for testing for COVID-19.

[14] A method for detecting SARS-COV-2 virus, comprising performing an amplification reaction through a LAMP method using the primer set of any one of [7] to [9] or the kit of any one of to [13].

[15] A method for testing for COVID-19, comprising detecting amplification of a target nucleic acid region of SARS-COV-2 virus using the primer set of any one of [7] to [7] or the kit of any one of to to test for presence or absence of infection with SARS-COV-2 virus.

### Advantageous Effects of Invention

According to the present invention, SARS-COV-2 can be promptly detected with high sensitivity by producing an oligonucleotide primer that is selectively hybridized with a sequence specific to SARS-COV-2 and amplifying the sequence specific to SARS-COV-2 through a LAMP method.

### DESCRIPTION OF EMBODIMENTS

Examples of samples used in the present invention include specimens, such as sputum, bronchoalveolar lavage fluid, nasal mucus, nasal suction fluid, nasal lavage fluid, nasal swabs, pharyngeal swabs, mouthwash, saliva, blood, serum, plasma, cerebrospinal fluid, urine, feces, and tissue, derived from humans or other animals suspected of being infected with SARS-COV-2 virus. In addition, cells used in infection experiments or the like or their culture solutions, or specimens derived from living bodies or specimens containing viruses isolated from cultured cells or the like can also be used as samples. These samples may be subjected to pretreatment such as separation, extraction, concentration, and purification.

Amplification of nucleic acids contained in a sample is achieved by a loop-mediated isothermal amplification method (WO 00/28082) called a LAMP method that is a nucleic acid amplification method which was developed by Notomi et al. and does not require temperature control indispensable for a PCR method. This method is a nucleic acid amplification method which enables an isothermal complementary strand synthesis reaction by combining primers which anneal their own 3′ terminals to template nucleotides to serve as starting points for complementary strand synthesis and are annealed to loops formed at this time. In addition, in the LAMP method, since the 3′ terminal of a primer is always annealed to a region derived from a sample, a check mechanism due to a complementary bond of sequences functions repeatedly. As a result, the LAMP method enables a nucleic acid amplification reaction with high sensitivity and high specificity.

Regarding oligonucleotide primers used in a LAMP method, there are at least 4 types of primers recognizing a sequence in a total of 6 regions of the sequence of a template nucleic acid, that is, regions F3c, F2c, and F1c from the 3′ terminal side and regions B3, B2, and B1 from the 5′ terminal side, which are called a forward inner primer, a backward inner primer, a forward outer primer, and a backward outer primer. In addition, complementary sequences of F1c, F2c, and F3c are respectively called F1, F2, and F3, and complementary strands of B1, B2, and B3 are respectively called B1c, B2c, and B3c. An inner primer is an oligonucleotide which recognizes a “specific nucleotide sequence region” on a target base sequence, has a sequence, providing a starting point for synthesis, at the 3′ terminal, and simultaneously has a sequence complementary to an arbitrary region of a nucleic acid synthesis reaction product with this primer as a starting point, at the 5′ terminal. Here, a primer containing a “sequence selected from F2” and a “sequence selected from F1c” is called a forward inner primer (hereinafter, an FIP primer), and a primer containing a “sequence selected from B2” and a “sequence selected from B1c” is called a backward inner primer (hereinafter, a BIP primer). On the other hand, an outer primer is an oligonucleotide which recognizes a “specific nucleotide sequence region present on the 3′ terminal side of a “specific nucleotide sequence region” on a target sequence and has a sequence providing a starting point for synthesis. Here, a primer containing a “sequence selected from F3” is called a forward outer primer (hereinafter, an F3 primer), and a primer containing a “sequence selected from B3” is called a backward outer primer (hereinafter, a B3 primer). Here, F in each primer represents a primer which complementarily binds to a sense strand of a target sequence and provides a starting point for synthesis, while B represents a primer which complementarily binds to an antisense strand of a target sequence and provides a starting point for synthesis. Here, the length of an oligonucleotide used as a primer is greater than or equal to 10 bases and preferably greater than or equal to 15 bases, and may be either chemically synthesized or natural. Each primer may be a single oligonucleotide or may be a mixture of a plurality of oligonucleotides.

In a LAMP method, in addition to the inner primers and the outer primers, another primer, that is, a loop primer, can be used. A loop primer is a primer having a sequence complementary to a sequence of a single-stranded portion of a loop structure on the 5′ terminal side of a dumbbell structure. When this primer is used, the number of starting points for nucleic acid synthesis increases, whereby the reaction time can be reduced and the detection sensitivity can be increased (WO 02/024902). A sequence of a loop primer may be a sequence of a target gene or selected from a complementary strand thereof, or may be other sequences as long as it is complementary to the above-described sequence of a single-stranded portion of a loop structure on the 5′ terminal side of a dumbbell structure. In addition, the loop primer may be one kind or two or more kinds, and is called a forward loop primer (hereinafter, LF) and a backward loop primer (hereinafter, LB) in the present specification.

In a case where a gene of a single-stranded RNA virus is detected through amplification by a LAMP method, an outer primer can be further added to increase the efficiency of a reverse transcription reaction in an initial stage of the amplification reaction. Since SARS-COV-2 is a plus-strand single-stranded RNA virus, an outer primer (B4 primer) may be added to the downstream side of a B3 primer.

SARS-COV-2 is an RNA virus. In a LAMP method, in a case where RNA is used as a template, a nucleic acid amplification reaction can similarly proceed by adding a reverse transcriptase to a reaction solution in a case where DNA is used as a template (RT-LAMP method).

The present inventors have conducted extensive studies on base sequences of primers in a LAMP method capable of promptly amplifying a sequence specific to SARS-COV-2, and a combination thereof, and as a result, they have designed primer sets from an N gene region and an RNA-dependent RNA polymerase (RdRP) gene region of a sequence (SEQ ID NO: 1) of SARS-CoV-2. That is, the present invention is a primer set consisting of at least one designed from an N gene region (a region from a 28976th base to a 29211th base of SEQ ID NO: 1) and an RNA-dependent RNA polymerase (RdRP) gene region (a region from a 15394th base to a 15595th base of SEQ ID NO: 1) of a sequence (SEQ ID NO: 1) of SARS-COV-2. The present invention is a primer set consisting of F3, B3, FIP, and BIP and designed as being a primer set comprising at least one selected from the following two sets a and b. In addition, the present invention is a primer set comprising an LF primer and an LB primer and comprising at least one selected from the following two sets a and b.

TABLE 1

| | | | SEQ |
|---|---|---|---|
| | Primer | | ID |
| | name | Base sequence | NO. |
| Primer | F3 | TCTGGTAAAGGCCAACAAC | 2 |
| set a | B3 | GCTGAAGCGCTGG | 3 |
| (N | FIP | AGTACGTTTTTGCCGAGGC- | 4 |
| gene) | | AAGGCCAAACTGTCACT | |
| | BIP | GTAACACAAGCTTTCGGCAG- | 5 |
| | | ATTAGTTCCTGGTCCCCAA | |
| | LF | AAGCCTCAGCAGCAGAT | 6 |
| | LB | TCCAGAACAAACCCAAGG | 7 |
| Primer | F3 | TTCTATAGATTAGCTAATGAGTGT | 8 |
| set b | B3 | ACTTATCGGCAATTTTGTTACC | 9 |
| (RdRP) | FIP | GAGGTTCCACCTGGTTTAACATAT- | 10 |
| | | CTCAAGTATTGAGTGAAATGGT | |
| | BIP | CAGGAGATGCCACAACTGCTTAT- | 11 |
| | | AGATAAAAGTGCATTAACATTGG | |
| | LF | GAACCGCCACACATGACC | 12 |
| | LB | CATTTGTCAAGCTGTCACGG | 13 |

In addition, the present inventors have conducted extensive studies on base sequences of primers in a LAMP method capable of promptly amplifying a base sequence specific to SARS-COV-2, and a combination thereof, and as a result, they have designed primer sets from an M gene region and an S gene region of a sequence (SEQ ID NO: 1) of SARS-COV-2. That is, the present invention is a primer set consisting of at least one designed from an M gene region (a region from a 26767th base to a 26977th base of SEQ ID NO: 1) and S gene region (a region from a 24660th base to a 24916th base of SEQ ID NO: 1) of a sequence (SEQ ID NO: 1) of SARS-COV-2. The present invention is a primer set which consists of F3, B3 (or B3 and B4), FIP, and BIP and consists of at least one selected from the following two sets c and d. In addition, the present invention is a primer set comprising an LF primer and an LB primer and comprising at least one selected from the following two sets c and d.

TABLE 2

| | Primer | |
|---|---|---|
| | name | Base sequence |
| Primer | F3 | TCGCAATGGCTTGTCTTG (SEQ ID NO: 14) |
| set c | B3 | GCAATACGAAGATGTCCACG (SEQ ID NO: |
| (M | | 15) |
| gene) | FIP | CGTACGCGCAAACAGT- |
| | | GGCTTGATGTGGCTCAG (SEQ ID NO: 16) |
| | BIP | CGTTCCATGTGGTCATTCA- |
| | | TAGAAGCGGTCTGGTCAG (SEQ ID NO: 17) |
| | LF | GAAAGAAGCAATGAAGTAGC (SEQ ID |
| | | NO: 18) |
| | LB | GAAACTAACATTCTTCTCAACG (SEQ ID |
| | | NO: 19) |
| Primer | F3 | TACTTGGACAATCAAAAAGAGTT (SEQ ID |
| set d | | NO: 21) |
| (S | B3 | GTCTGTAGTAATGATTTGTGGTTC (SEQ ID |
| gene) | | NO: 22) |
| | B4 | TGTCTGTAGTAATGATTTGTGG (SEQ ID |
| | | NO: 23) |
| | FIP | CTTTTCTTGTGCAGGGACA- |
| | | CTATCATCTTATGTCCTTCCCTC (SEQ ID |
| | | NO: 24) |
| | BIP | TCACAACTGCTCCTGCC- |
| | | TGAAACAAAGACACCTTCACG (SEQ ID |
| | | NO: 25) |
| | LF | ACATGCAAGAAGACTACACCA (SEQ ID |
| | | NO: 26) |
| | LB | GATGGAAAAGCACACTTTCCT (SEQ ID |
| | | NO: 27) |

Furthermore, the present invention is a kit comprising the above-described primer set c or d and a fluorescence labeling probe. A probe for the primer set c is a probe consisting of SEQ ID NO: 20, and a probe for the primer set d is a probe consisting of SEQ ID NO: 28. Cytosine bases at the 3′ terminals of these probes are fluorescently labeled with BODIPY (registered trademark) FL dye. These probes are Q-probes. That is, when the probes are bound to target nucleic acids, fluorescent dyes and guanine bases of the target nucleic acids are in close proximity, and the fluorescence intensity of the fluorescent dyes is reduced by a quenching action of the guanine base.

TABLE 3

| | Base sequence |
|---|---|
| Probe for | ATCCAGAAACTAACATTCTTCTCAACGTGCCACTCC- |
| primer | bodipyFL (SEQ ID NO: 20) |
| set c | |
| Probe for | GCAAGAAGACTACACCATGAGGTGCTGAC-bodipyFL |
| primer | (SEQ ID NO: 28) |
| set d | |

A method for detecting SARS-COV-2 virus of the present invention is a method for performing an amplification reaction through a LAMP method using the primer set of the present invention.

Enzymes used in nucleic acid synthesis are not particularly limited as long as these are template-dependent nucleic acid synthase having a strand substitution activity. Examples of such enzymes include Bst DNA polymerase (large fragment), Bca (exo-) DNA polymerase, and Klenow fragment of Escherichia coli DNA polymerase I, and preferably include Bst DNA polymerase (large fragment).

Reverse transcriptases used in an RT-LAMP method are not particularly limited as long as these are enzymes having an activity of synthesizing DNA using RNA as a template. Examples of such enzymes include reverse transcriptases derived from AMV, Cloned AMV, or MMLV, Superscript II, ReverTra Ace, and Thermoscript, and preferably include reverse transcriptases derived from AMV or Cloned AMV. In addition, when an enzyme, such as Bca DNA polymerase, having both a reverse transcriptase activity and a DNA polymerase activity is used, the RT-LAMP reaction can be performed with one enzyme.

Enzymes or reverse transcriptases used in nucleic acid synthesis may be purified from viruses, bacteria, or the like or may be produced by a gene recombination technique. In addition, these enzymes may be modified through, for example, fragmentation or substitution with an amino acid.

A method for testing for COVID-19 of the present invention is a method for detecting amplification of a target nucleic acid region of SARS-COV-2 virus using the primer set of the present invention to test for presence or absence of infection with SARS-COV-2 virus. Well-known techniques can be applied to detection of nucleic acid amplification products after a LAMP reaction. For example, the detection can be easily performed through a method in which a labeled oligonucleotide that specifically recognizes an amplified sequence or a fluorescent intercalator method (JP 2001-242169) is used or through subjecting a reaction solution after the completion of a reaction directly to agarose gel electrophoresis. In agarose gel electrophoresis, a large number of bands with different base lengths are detected in a ladder shape in LAMP amplification products. In addition, since insoluble magnesium pyrophosphate which is produced through a synthetic reaction of nucleic acids is produced in a LAMP method, a reaction solution is cloudy to the extent that it can be visually observed. A nucleic acid amplification reaction can also be detected by optically measuring such cloudiness (WO 01/83817). Furthermore, calcein, a metal indicator, can also be added to a reaction solution to detect a change in concentration of metal ions as a change in fluorescence with the progress of an amplification reaction (JP 2004-283161).

In a method for diagnosing COVID-19, the primer set of the present invention can be pre-packaged into a kit together with various reagents necessary for detecting nucleic acid amplification. Specifically, various oligonucleotides required as the primers or the loop primers of the present invention, 4 kinds of dNTPs as substrates for nucleic acid synthesis, DNA polymerases for performing nucleic acid synthesis, reverse transcriptases, buffer solutions or salts that provide suitable conditions for an enzymatic reaction, protective agents that stabilize enzymes or templates, and reagents required for detecting reaction products as necessary are provided as a kit.

### EXAMPLES

Hereinafter, the present invention will be described specifically with reference to examples. However, the present invention is not limited by these examples.

### Example 1. Confirmation of Detection Sensitivity of Primer Sets a and b

The detection sensitivity of a LAMP method was confirmed.

### 1. Preparation of Sample and Reagent

### 1) Sample (Transcribed RNA)

An RNA template was prepared by integrating a plasmid with cDNA prepared through RT-PCR from a SARS-COV-2 gene and transcribing and purifying RNA from the plasmid DNA. Script Max (registered trademark) Thermo T7 Transcription Kit (manufactured by TOYOBO Co., LTD., Code Number: TSK-101) was used for the transcription, and RNeasy (registered trademark) Mini Kit (manufactured by QIAGEN N.V., Catalog Number: No. 74104) was used for RNA purification. Diluted solutions of 10 copies to 10³ copies per microliter were prepared from the purified RNA and used as sample solutions. In addition, a yeast RNA solution was used as a sample solution of 0 copies (negative control).

2) Concentration and Composition of Reagent Used in LAMP Method 25 μL of a LAMP reaction reagent having following composition was prepared in a 0.2 mL reagent tube. As a primer, the primer set a or b shown in Table 1 was used.
20 mM Tricine (pH 8.6),
30 mM KCl,
8 mM MgSO₄,
1.4 mM dNTPs,
0.5% Tween 20,
1.6 mM DTT,
1.6 μM FIP Primer and BIP Primer,
0.2 μM F3 Primer and B3 Primer,
0.8 μM Loop Primer F (LF) and Loop Primer B (LB),
AMV Reverse Transcriptase 1.0 U (20 U/μL, Manufactured by Roche Molecular Systems, Inc.),
Bst DNA polymerase 22.8 U (Manufactured by New England Biolabs),
RNase Inhibitor (40 U/μL, Manufactured by Promega) 1 μL, and
RNA template (100 copies) 5 μL.

### 2. Reaction by Nucleic Acid Amplification Method

### 1) Reaction by LAMP Method

1 μL of each of sample solutions containing 0 or 10 to 10³ copies of the target sequence were added to the LAMP reaction reagent prepared in 2) of 1 to obtain 25 μL of each final reaction solution. A real-time turbidity measurement device LoopampEXIA (registered trademark) was used for measurement, and the reaction conditions of 63° C. and 60 minutes were set.

### 3. Measurement Results

Measurement results for each of the primer sets are shown in Table 4. It was found that the number of copies that can be detected was up to 10 in the primer sets a with the N gene as a detection target and up to 50 in the primer sets b with the RdRP gene as a detection target.

TABLE 4
N Gene Use of transcribed RNA
Detection time: Minute

| | Primer set a | | | |
|---|---|---|---|---|
| Number | | | Average | |
| of copies | 1 | 2 | value | S.D. |
| 1,000 | 16.4 | 16.2 | 16.3 | 0.14 |
| 500 | 16.5 | 16.7 | 16.6 | 0.14 |
| 200 | 18.0 | 17.7 | 17.9 | 0.21 |
| 100 | 18.4 | 17.6 | 18.0 | 0.57 |
| 50 | 23.3 | 20.2 | 21.8 | 2.19 |
| 20 | 20.1 | 23.0 | 21.6 | 2.05 |
| 10 | 25.0 | 37.3 | 31.2 | 8.70 |
| Negative | Not | Not | | |
| control | detected | detected | | |

RdRP Gene Use of transcribed RNA
Detection time: Minute

| | Primer set b | | | |
|---|---|---|---|---|
| Number | | | Average | |
| of copies | 1 | 2 | value | S.D. |
| 1,000 | 17.6 | 17.6 | 17.6 | 0.00 |
| 500 | 19.2 | 18.3 | 18.8 | 0.64 |
| 200 | 22.8 | 17.7 | 20.3 | 3.61 |
| 100 | 26.5 | 21.6 | 24.1 | 3.46 |
| 50 | 24.6 | 26.0 | 25.3 | 0.99 |
| 20 | Not | Not | | |
| | detected | detected | | |
| 10 | 28.2 | Not | 28.2 | |
| | | detected | | |
| Negative | Not | Not | | |
| control | detected | detected | | |

### Example 2. Detection Sensitivity When Genomic RNA Was Used as Template

Viral genomic RNA dispensed from the National Institute of Infectious Disease was used to examine the detection sensitivity. Concurrently, PCR was also carried out for comparison of the detection sensitivity therebetween.

### 1. Preparation of Sample and Reagent

### 1) Sample (Genomic RNA)

Diluted solutions of 50 copies to 1.6×10³ copies of genomic RNA of SARS-CoV-2 dispensed from the National Institute of Infectious Disease per microliter were prepared used as sample solutions.

### 2) Concentration and Composition of Reagent Used in LAMP Method

A reaction reagent was prepared in the same manner as in Example 1. The primer set a with the N gene as a detection target was used as a primer set.

### 3) Concentration and Composition of Reagent Used in PCR

An amplification reagent and a primer with the N gene as a detection target were prepared in accordance with “Pathogen Detection Manual 2019-nCOV Ver.2.7” of the National Institute of Infectious Disease.

### 2. Reaction by Nucleic Acid Amplification Method

### 1) Reaction by LAMP Method

An amplification reaction was carried out with a real-time turbidity measurement device LoopampEXIA (registered trademark) in the same manner as in Example 1 at 63° C. for 60 minutes.

### 2) Reaction by PCR

An RT-PCR method in which TaqMan probe was used was carried out in accordance with “Pathogen Detection Manual 2019-nCOV Ver.2.7” of the National Institute of Infectious Disease.

### 3. Measurement Results

The measurement results in each method are shown in Table 5. Although genomic RNA up to 50 copies was able to be detected in both amplification methods, the detection in the LAMP method was able to be performed in a shorter period of time than PCR.

TABLE 5

| Measurement results for Genomic RNA | | | | | |
|---|---|---|---|---|---|
| Detection time: Minute | | | | | |
| | LAMP Method | | | | |
| Number | | | | Average | |
| of copies | 1 | 2 | 3 | value | S.D. |
| 1,600 | 20.3 | 19.7 | 19.3 | 19.8 | 0.5 |
| 800 | 20.0 | 19.6 | 20.2 | 19.9 | 0.3 |
| 400 | 21.2 | 21.9 | 21.6 | 21.6 | 0.4 |
| 200 | 23.2 | 25.0 | 22.7 | 23.6 | 1.2 |
| 100 | 24.7 | 26.6 | 23.1 | 24.8 | 1.8 |
| 50 | 26.3 | 26.6 | 35.9 | 29.6 | 5.5 |
| Negative | Not | Not | Not | | |
| control | detected | detected | detected | | |

Detection time: Minute

| | PCR | | | | |
|---|---|---|---|---|---|
| Number | | | | Average | |
| of copies | 1 | 2 | 3 | value | S.D. |
| 1,600 | 29.5 | 29.6 | 29.6 | 29.6 | 0.1 |
| 800 | 30.5 | 30.7 | 30.6 | 30.6 | 0.1 |
| 400 | 31.5 | 31.5 | 31.5 | 31.5 | 0.0 |
| 200 | 32.7 | 32.5 | 32.6 | 32.6 | 0.1 |
| 100 | 33.2 | 33.5 | 34.2 | 33.6 | 0.5 |
| 50 | 34.6 | 34.7 | 35.0 | 34.8 | 0.2 |
| Negative | Not | Not | Not | | |
| control | detected | detected | detected | | |

### Example 3. Confirmation of Detection Sensitivity of Primer Sets c and d

The detection sensitivity of a LAMP method was confirmed.

### 1. Preparation of Sample and Reagent

### 1) Sample (Transcribed RNA)

Artificial genes in which sequences of an M region and an S region were respectively incorporated were synthesized, and transcribed RNA copies thereof were used as templates. Synthesis of the artificial genes was outsourced to Eurofins. The sequences of the artificial genes are represented by SEQ ID NO: 29 (artificial gene for detecting M region), and SEQ ID NO: 30 (artificial gene for detecting S region).

### 2) Concentration and Composition of Reagent Used in LAMP Method

A LAMP reaction reagent having the following composition was prepared.
LAMP Reaction reagent:
20 mM Tricine (pH 8.6),
30 mM KCl,
0.1% Tween 20,
1.4 mM dNTPs,
8 mM MgSO₄,
1.6 mM DTT,
PPase 20 mU
Bst DNA polymerase 25 U,
RNase Inhibitor 1.0 U, and
AMV Reverse Transcriptase 1.0 U.

A primer set c or d, a fluorescence labeling probe corresponding to the primer set, and SYTO™ 63 Red Fluorescent Nucleic Acid Stain were added to the reaction reagent, a master mix (MM) prepared with a purified water (DW) to have a concentration of 15 μL/test was used, and a total of 25 μL including 5 μL of DW, 5 μL of a template, and the master mix was used in one reaction. The reaction reagent was prepared on ice.
Master mix (amount per reaction):
LAMP Reaction reagent 12.5 μL
5 μM SYTO 0.5 μL
10 μM Probe 0.1 μL
Primer set Appropriate amount
DW Total of 15 μL.
Amount per reaction:
Master mix 15 μL
DW 5 μL
RNA template 5 μL.

### 2. Reaction by Nucleic Acid Amplification Method

### 1) Reaction by LAMP Method

A real-time quantitative PCR system LightCycler (registered trademark) 96 (manufactured by Roche Molecular Systems, Inc.) was used for measurement, and evaluation was performed at a reaction temperature of 63° C.

### 3. Measurement Results

Measurement results for each of the primer sets are shown in Table 6.

TABLE 6
Detection time: Minute

| Number of | Primer set c | | | | |
|---|---|---|---|---|---|
| copies | 1 | 2 | 3 | 4 | Average |
| 1,000 | 6.4 | 6.5 | — | — | 6.5 |
| 50 | 12.6 | 9.6 | 10.3 | 8.9 | 10.4 |
| 25 | 10.2 | 10.8 | 9.0 | 13.1 | 10.8 |
| 12.5 | 9.8 | 12.6 | 19.3 | 11.7 | 13.4 |
| Negative | Not | Not | — | — | — |
| control | detected | detected | | | |

Detection time: Minute

| Number of | Primer set d | | | | | |
|---|---|---|---|---|---|---|
| copies | 1 | 2 | 3 | 4 | 5 | Average |
| 1,000 | 5.9 | 5.4 | 5.7 | 5.8 | 6.0 | 5.8 |
| 100 | 6.9 | 7.8 | 6.5 | 8.2 | 7.4 | 7.4 |
| 50 | 7.4 | 8.2 | 8.9 | 8.1 | 8.1 | 8.1 |
| 25 | 9.8 | 10.0 | 12.7 | 8.3 | 8.8 | 9.9 |
| 12.5 | Not | 12.2 | Not | 10.0 | 18.1 | 13.4 |
| | detected | | detected | | | |
| Negative | Not | Not | Not | Not | Not | — |
| control | detected | detected | detected | detected | detected | |

The minimum detection sensitivity that could be detected within 15 minutes was 25 copies/test in any system in which any primer set was used.

### Example 2. Confirmation of Cross-Reactivity with SARS Virus

At GGGenome (https://gggenome.dbcls.jp/ja/), cross-reactivity with 38 kinds of respiratory system-related bacteria and pathogenic microorganisms (Human coronavirus 229E, Human coronavirus OC43, Human coronavirus HKUI, Human coronavirus NL63, SARS-coronavirus, SARS-coronavirus-2, MERS-coronavirus, Adenovirus, Human Metapneumovirus, Parainfluenza virus 1-4, Influenza A, Influenza B, Enterovirus, Respiratory syncytial virus, Rhinovirus, Chlamydia pneumonia, Haemophilus influenzae, Legionella pneumophila, Mycobacterium tuberculosis, Streptococus pneumonia, Streptococcus pyrogenes, Bordetella pertussis, Mycoplasma pneumoniae, Pneumocystis jirovecii (PJP), Influenza C, Parechovirus, Candida albicans, Corynebacterium diphtheriae, Legionella non-pneumophila, Bacillus anthracosis (Anthrax), Moraxella cararrhalis, Neisseria elongate and miningitidis, Pseudomonas aeruginosa, Staphylococcus epidermis, Staphylococcus salivarius, Leptospirosis, Chlamydia psittaci, Coxiella burneti (Q-Fever), Streptococcus aureus) was confirmed in silico for each of the M region and the S region.

As a result, in only an M region of SARS-Coronavirus (SARS virus), a mismatch in a probe design region was within 5 bases and each of mismatches in F1, F2, B1, and B2 primer design regions was within 3 bases, and these became positions for a LAMP reaction. Therefore, the cross-reactivity with SARS virus was confirmed.

Transcribed RNA of an artificial gene with which a sequence of SARS virus (GenBank NC004718.3) having high homology for each region was integrated was used as a template of the SARS virus (synthesis request destination: Eurofins Scientific). The amount of template was 1.0×10⁷ copies/test.

Measurement results for each of the primer sets are shown in Table 7.

TABLE 7
Detection time: Minute

| | Primer set c | | | | |
|---|---|---|---|---|---|
| Template | 1 | 2 | 3 | 4 | Average |
| 1 × 10⁷ | Not | Not | Not | Not | — |
| Copies of | detected | detected | detected | detected | |
| SARS | | | | | |
| 1,000 Copies | 5.6 | 5.6 | — | — | 5.6 |
| of SARS- | | | | | |
| CoV-2 | | | | | |
| Negative | Not | Not | — | — | — |
| control | detected | detected | | | |

Detection time: Minute

| | Primer set d | | | | |
|---|---|---|---|---|---|
| Template | 1 | 2 | 3 | 4 | Average |
| 1 × 10⁷ | Not | Not | Not | Not | — |
| Copies of | detected | detected | detected | detected | |
| SARS | | | | | |
| 1,000 Copies | 5.7 | 5.7 | — | — | 5.7 |
| of SARS- | | | | | |
| CoV-2 | | | | | |
| Negative | Not | Not | — | — | — |
| control | detected | detected | | | |

No cross-reaction with SARS virus was observed in both detection systems. In addition, assuming a case of co-infection with SARS-COV-2 and SARS virus, tests in which 1.0×10⁶ copies/test of transcribed RNA of SARS virus was mixed with 25 or 1,000 copies/test of transcribed RNA of SARS-COV-2 were performed.

Measurement results for each of the primer sets are shown in Table 8.

TABLE 8
Detection time: Minute

| | | Primer set c | | | |
|---|---|---|---|---|---|
| Template | | 1 | 2 | 3 | Average |
| 1,000 | 1 × 10⁶ | 5.2 | 5.2 | 5.1 | 5.2 |
| Copies of | Copies of | | | | |
| SARS- | SARS | | | | |
| CoV-2 | No SARS | 5.2 | 5.2 | 5.3 | 5.2 |
| 25 Copies | 1 × 10⁶ | 6.7 | 7.9 | 7.2 | 7.3 |
| of SARS- | Copies of | | | | |
| CoV-2 | SARS | | | | |
| | No SARS | 6.9 | 7.5 | 6.9 | 7.1 |

| | | | | |
|---|---|---|---|---|
| Negative control | Not | Not | Not | — |
| | detected | detected | detected | |

Detection time: Minute

| | | Primer set d | | | |
|---|---|---|---|---|---|
| Template | | 1 | 2 | 3 | Average |
| 1,000 | 1 × 10⁶ | 5.4 | 5.5 | 5.4 | 5.4 |
| Copies of | Copies of | | | | |
| SARS- | SARS | | | | |
| CoV-2 | No SARS | 5.5 | 5.5 | 5.6 | 5.5 |
| 25 Copies | 1 x 10⁶ | 7.8 | 9.9 | 10.0 | 9.2 |
| of SARS- | Copies of | | | | |
| CoV-2 | SARS | | | | |
| | No SARS | 11.3 | 10.3 | 12.3 | 11.3 |

| | | | | |
|---|---|---|---|---|
| Negative control | Not | Not | Not | — |
| | detected | detected | detected | |

SARS-COV-2 could also be detected in the mixing test.

1. A primer set for detecting SARS-COV-2 virus through amplification by a LAMP method,
wherein the primer set consists of at least one designed from regions (a) and (b):
(a) a region from a 28976th base to a 29211th base of SEQ ID NO: 1; and
(b) a region from a 15394th base to a 15595th base of SEQ ID NO: 1.

2. The primer set of claim 1, which is a primer set for detecting SARS-COV-2 virus through amplification by a LAMP method,
wherein the primer set comprises at least one selected from the group consisting of (a1) and (b1):
(a1) a primer set consisting of an F3 primer consisting of SEQ ID NO: 2, a B3 primer consisting of SEQ ID NO: 3, an FIP primer consisting of SEQ ID NO: 4, and a BIP primer consisting of SEQ ID NO: 5; and
(b1) a primer set consisting of an F3 primer consisting of SEQ ID NO: 8, a B3 primer consisting of SEQ ID NO: 9, an FIP primer consisting of SEQ ID NO: 10, and a BIP primer consisting of SEQ ID NO: 11.

3. A primer set for detecting SARS-COV-2 virus through amplification by a LAMP method,
wherein the primer set consists of at least one selected from the group consisting of (a2) and (b2):
(a2) an LF primer consisting of SEQ ID NO: 6 and an LB primer consisting of SEQ ID NO: 7 in addition to the primer set of (a1) of claim 2; and
(b2) an LF primer consisting of SEQ ID NO: 12 and an LB primer consisting of SEQ ID NO: 13 in addition to the primer set of (b1) of claim 2.

4. A method for detecting SARS-COV-2 virus, comprising:
performing an amplification reaction through a LAMP method using the primer set of claim 1.

5. A method for testing for COVID-19, comprising:
detecting amplification of a target nucleic acid region of SARS-COV-2 virus using the primer set of claim 1 to test for presence or absence of infection with SARS-COV-2 virus.

6. A kit for testing for COVID-19, comprising the primer set of claim 1.
